(19) United States
(12) Patent Application Publication
(10) Pub. No.: US 2025/0344816 A1
(43) Pub. Date: Nov. 13, 2025

(54) NEEDLE WITH HEEL BYPASS

(71) Applicant: Industrial Strength, LLC, Wallingford, CT (US)

(72) Inventor: Maria Pinto, Wallingford, CT (US)

(21) Appl. No.: 19/200,060

(22) Filed: May 6, 2025

Related U.S. Application Data
(60) Provisional application No. 63/643,666, filed on May 7, 2024.

(51) Int. Cl. A44C 7/00; A61B 17/34

(52) U.S. Cl. CPC A44C 7/001; A61B 17/3417; A61B 2017/3454

(57) ABSTRACT

A needle with an elongated body with a proximal end, a distal end, and a hollow channel, the proximal end configured to penetrate tissue, wherein the elongated body has an upper wall with an inner surface and an outer surface and a bottom wall with an inner surface and an outer surface. The proximal end has a beveled face and an opening within the beveled face in communication with the hollow channel, wherein the beveled face terminates at a piercing tip at its proximal end and has a bevel bottom at its distal end formed by an edge of the outer surface of the top wall. The beveled face has a heel formed by an edge of the inner surface of the top wall delimiting the opening and a bend positioned between the heel and the bevel bottom such that the piercing tip is positioned above the heel.

### FIELD OF THE TECHNOLOGY

The present technology relates to a needle for use in medical cosmetology or medical procedures and a method for manufacturing the needle. In particular, the present technology relates to a needle with a strategic bend and a heel bypass. The present invention also relates to a bending process for needles used in body piercing, and in particular, on a bending process to achieve a heel bypass bend to enhance the performance and functionality of the needles.

### BACKGROUND OF THE INVENTION

The field of medical cosmetology includes body piercing, which is a form of body modification that involves the practice of puncturing a part of the human body, creating an opening in which jewelry may be worn. Commonly used modern body piercing methods involve making an opening using a beveled-tip hollow medical needle, which is available in different lengths, gauges and even shapes. While straight needles are useful for many body parts, curved needles are manufactured for areas where straight needles are not ideal. The needle selected is typically the same gauge (or sometimes larger as with cartilage piercings) as the initial jewelry to be worn, with higher gauges indicating thinner needles. The needle is inserted into the body part being pierced, frequently by hand but sometimes with the aid of a needle holder or pusher. While the needle is still in the body, the initial jewelry to be worn in the piercing is pushed through the opening, following the back of the needle. Jewelry is often inserted into the hollow end of a needle, so that as the needle pulls through, the jewelry is left behind.

However, conventional piercing needle designs have a number of shortcomings. For example, existing needles often cause unnecessary tissue trauma due to the heel of the needle cutting into the tissue during piercing. The heel of the bevel often poses challenges for piercers, necessitating precise handling to avoid interference during piercing. The piercing community has developed bevel theory, guiding piercers on proper needle manipulation to minimize discomfort for clients. However, these methods still involve inherent limitations due to the placement of the heel. Additionally, users typically need to hold the needle at an angle to ensure straight piercing, which can be challenging and may compromise procedural outcomes.

The objective of the present invention is to address the shortcomings of the known needle designs. In particular, there is a need to develop a needle that is capable of accurately piecing the tissue with the needle positioned perpendicular to the tissue without the need for precise angling of the needle to achieve a straight cut. There is also a need to develop a needle that can pierce tissue with minimal resistance and no extra effort needed to compensate for resistance resulting in an easier piercing procedure and less pain experienced during the procedure.

### SUMMARY OF THE INVENTION

The invention introduces an innovative needle design characterized by a strategic bend in the heel and primary bevel region, facilitating seamless tissue bypass during piercing procedures. This bend effectively places the heel of the needle below the cutting edge and aligns the secondary cutting edge with the cannula tubing. The primary goal is to mitigate tissue damage caused by the heel of the needle, while also enabling straight piercing without necessitating the needle to be held at an angle.

The present invention further addresses the shortcomings by introducing a novel bending process that strategically positions the heel of the needle below the cutting edge, widens the base of the heel and primary bevel region to effectively bypass, and aligns the cutting edge with the top of the cannula tubing. This process is achieved using an exemplary manufacturing equipment unit, along with appropriate tooling and programming, to bend needles to specific degrees based on their outer diameter and inner diameter. The novel design also includes a star tip that creates a flatter cutting surface, optimizing both entry and exit through tissue, which results in a smoother, more controlled transition for improved sharpness.

In one aspect, a needle is provided having an elongated body with a proximal end, a distal end, and a hollow channel extending between the proximal and distal ends, the proximal end configured to penetrate tissue, wherein the elongated body has an upper wall with an inner surface and an outer surface and a bottom wall with an inner surface and an outer surface. The proximal end has a beveled face and an opening within the beveled face in communication with the hollow channel, wherein the beveled face terminates at a piercing tip at its proximal end and has a bevel bottom at its distal end formed by an edge of the outer surface of the top wall. The beveled face has a heel formed by an edge of the inner surface of the top wall delimiting the opening, and incudes a bend positioned between the heel and the bevel bottom such that the piercing tip is positioned above the heel.

In some embodiments, the piercing tip is aligned with a longitudinal axis of the outer surface of the top wall of the elongated body.

In some embodiments, the beveled face further includes a pair of opposing side bevels positioned proximally of the heel, the side bevels intersecting at the piercing tip, wherein a top surface of the side bevels is at an angle to a top surface of the beveled face.

In certain embodiments, the bend has an angle α between a longitudinal axis of the bottom wall of the elongated body distal to the bend and a longitudinal axis of the bottom wall of the elongated body proximal to the bend, wherein a is between about 1 to about 24 degrees.

In some embodiments, the needle also includes a cannula positioned over the elongated body.

The needle may be configured for a medical cosmetology procedure.

In some embodiments, the bend is positioned at the heel. In additional embodiments, the bend is positioned at the bevel bottom.

In another aspect of the invention, a needle includes an elongated body with a proximal end, a distal end, a substantially cylindrical wall, and a hollow channel extending between the proximal and distal ends, the proximal end configured to penetrate tissue. The proximal end has a beveled face and an opening within the beveled face in communication with the hollow channel, wherein the beveled face terminates at a piercing tip at its proximal end. The beveled face has a pair of opposing side bevels intersecting at the piercing tip, wherein a top surface of the side bevels is at an angle to a top surface of the beveled face. The needle further includes a star tip positioned adjacent the piercing tip and comprising a top surface extending in the same plane as the top surface of the beveled face and at an angle to a top surface of the side bevels.

In some embodiments, the top surface of the star tip is substantially flat.

In certain embodiments, the beveled face terminates at a piercing tip at its proximal end and has a bevel bottom at its distal end formed by an edge of an outer surface of the substantially cylindrical wall, the beveled face having a heel formed by an edge of an inner surface of the substantially cylindrical wall delimiting the opening, and the beveled face including a bend positioned between the heel and the bevel bottom such that the piercing tip is positioned above the heel.

In some embodiments, the angle between the top surface of the side bevels and the top surface of the beveled face is in a range of about 1 degree to about 24 degrees.

In yet another embodiment, a needle has an elongated body with a wall and a hollow channel extending therethrough, a beveled face with an opening within the beveled face in communication with the hollow channel, wherein the beveled face terminates at a piercing tip at its proximal end and has a bevel bottom at its distal end formed by an edge of an outer surface of the wall, a pair of opposing side bevels intersecting at the piercing tip, wherein a top surface of the side bevels is at an angle to a top surface of the beveled face, a heel formed by an edge of an inner surface of the wall delimiting the opening, a star tip adjacent the piercing tip having a top surface extending in the same plane as the top surface of the beveled face and at an angle to a top surface of the side bevels, and a bend positioned between the heel and the bevel bottom such that the heel is positioned below the piercing tip.

In some embodiments, the piercing tip is aligned with a longitudinal axis of the outer surface of the wall.

In certain embodiments, the bend has an angle α between a longitudinal axis of the wall distal to the bend and a longitudinal axis of the wall proximal to the bend, wherein a is between about 1 degree to about 24 degrees.

In some embodiments, the elongated body has a widening at the bend.

In certain embodiments, the piercing tip is aligned with a longitudinal axis of the outer surface of the wall opposite the piercing tip.

Further objects, aspects, features, and embodiments of the present technology will be apparent from the drawing figures and below description.

### BRIEF DESCRIPTION OF THE DRAWINGS

FIG. 1 shows a perspective view of a prior art needle design.

FIG. 2 shows a side view of the prior art needle design.

FIG. 3 shows a perspective view of the needle design according to an exemplary embodiment of the present invention.

FIG. 4 shows a side view of the needle design according to an exemplary embodiment of the present invention.

FIG. 5 shows an enlarged view of the heel to bottom of bevel area on FIG. 3.

FIG. 6 shows a top view of the needle of FIG. 3.

FIG. 7 shows a perspective view of an exemplary embodiment of the needle bevel design in accordance with the present invention.

FIG. 8 shows a top view of the needle of FIG. 7.

FIG. 9 shows a top view of the needle design according to an exemplary embodiment of the present invention.

FIG. 10 shows manufacturing equipment for making the needle design according to an exemplary embodiment of the technology.

FIG. 11A shows a partial view of the manufacturing equipment of FIG. 10, showing a needle fixture holder being inserted.

FIG. 11B shows a partial side view of the die and punch of FIG. 11A during the needle bending process.

FIG. 11C shows a partial enlarged view of the manufacturing equipment of FIG. 10, showing the needle fixture holder positioned in the equipment.

### DETAILED DESCRIPTION OF THE INVENTION

The following detailed description is merely exemplary in nature and is not intended to limit the disclosed invention or any associated methods for producing or using the same described herein. Furthermore, there is no intention to be bound by any theory presented in the preceding background or the following detailed description.

It is noted that, as used in the specification and the claims, the singular form “a,” “an,” and “the” comprises plural referents unless the context clearly indicates otherwise. For example, reference to a component in the singular is intended to comprise a plurality of components.

The term “about” is to be construed as modifying a term or value such that it is not an absolute. This term will be defined by the circumstances. This includes, at the very least, the degree of expected experimental error, technique error and instrument error for a given technique used to measure a value. In general, this term used in connection with a numerical value throughout the specification and the claims denotes an interval of accuracy, familiar and acceptable to a person skilled in the art. In general, such interval of accuracy is +10%. Thus, “about ten” means 9 to 11. All numbers in this description indicating amounts, ratios of materials, physical properties of materials, or use are to be understood as modified by the word “about,” except as otherwise explicitly indicated.

“At least one”, as used herein, relates to one or more, i.e., 1, 2, 3, 4, 5, 6, 7, 8, 9, or more.

The term “comprising” and “comprises” is synonymous with “including,” “having,” “containing,” or “characterized by.” These terms are inclusive and open-ended and do not exclude additional, unrecited elements or method steps.

The present technology relates to a needle with a strategic bend and a heel bypass. The inventive needle positions the heel of the needle below the cutting edge and aligns the secondary cutting edge with the cannula tubing. This inventive design prevents tissue damage caused by the heel of the needle while ensuring straight piercing without the need for holding the needle at an angle.

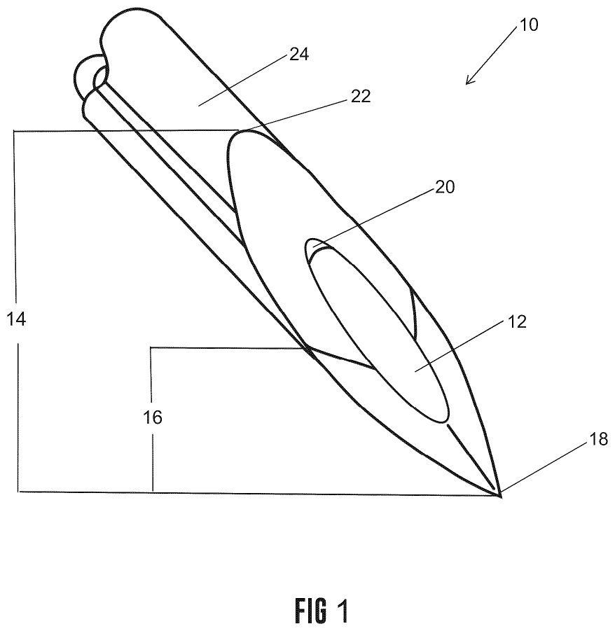

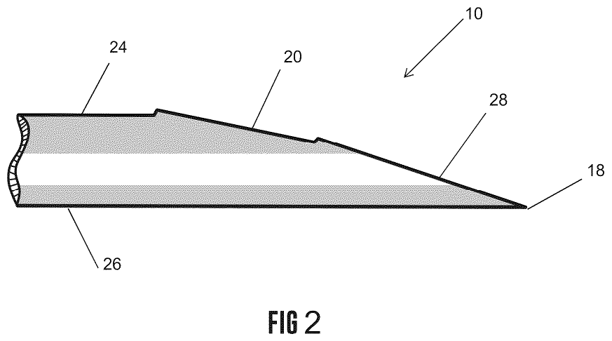

FIGS. 1-2 illustrate a convention piercing needle design 10. Piercing needles are typically made from hollow tubes called needle blanks. From these tubes, a machine grinds down the sharp ends of the needle to create a piercing channel 12. The needle tubing has an outer dimension (OD) and inner dimension (ID) that create a nominal wall thickness. The needle has a top wall 24 and a bottom wall 26, with the hollow piercing channel 12 inside. As seen in FIGS. 1-2, there is a first (primary) bevel 14 that extends from a bottom of bevel 22 to a piercing tip 18. There is also a second (secondary) bevel 16 positioned in the mid-section of the needle and extending from a mid-section 28 to the piercing tip 18. The piercing tip 18 is the first part of the needle that enters the skin and creates the initial small puncture in the skin. The secondary bevel 16 is considered a cutting bevel, which creates a crescent or C-shaped cut through the skin. The first bevel 14 is considered a stretching bevel, which takes that crescent cut and stretches it up into a round or O-shaped channel by displacing the tissue up and around the needle.

The first bevel 14 has a heel 20 defined by an edge of an inner wall of the top wall 24 of the needle. As seen in FIG. 2, the heel 20 is typically positioned above the cutting edge 18. Because of this design, during the piercing operation, as the needle 10 enters a subject's skin after it is initially pierced by the piercing tip 18, the heel 20 may contact the skin and create a secondary cut. This may cause additional bleeding, pain and discomfort to the subject during the piercing process. The present invention is designed to overcome these drawbacks of the conventional piercing needles.

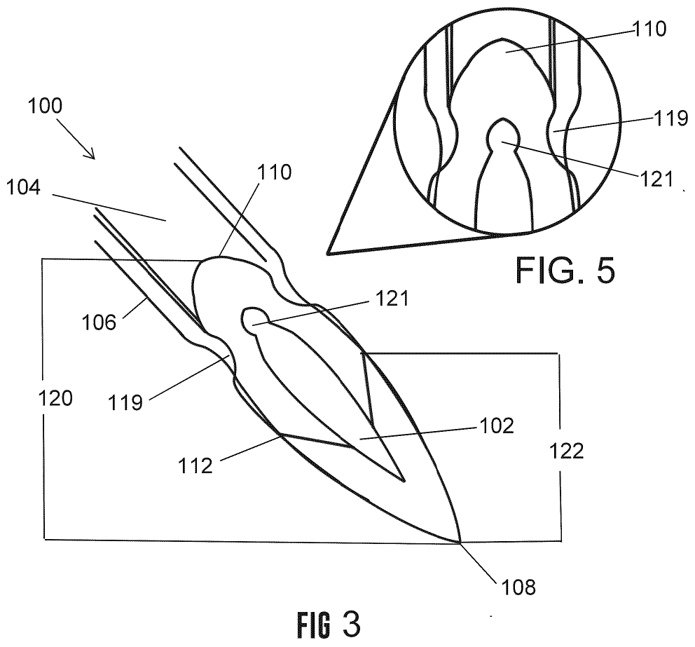

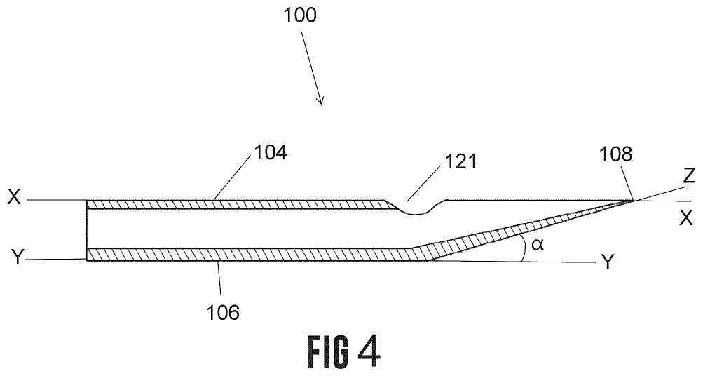

One exemplary embodiment of the present technology is illustrated in FIGS. 3-4. A needle 100 comprises a substantially cylindrical wall defining a longitudinal passage or a hollow piercing channel 102. The needle tubing has a wall with an outer diameter (OD) and an inner diameter (ID) creating a wall thickness. The wall has a top wall 104 terminating at a bottom of bevel 110 and a bottom wall 106 terminating at a piercing tip 108, as shown in FIG. 4. The needle may be made with any suitable material, such as stainless steel, titanium, plastic or other suitable materials known in the art. The needle 100 may be used alone or together with a cannula that is inserted over the needle and is inserted into a subject's skin together with the needle. Once the skin is pierced, the needle may be withdrawn, and the cannula remains in the pierced opening and is used to insert and guide a decorative article through a hollow cannula channel. The needle 100 may be used alone or together with a needle blank and/or a transfer pin that is inserted inside the needle. Once the skin is pierced, the needle may be withdrawn using a transfer pin option to guide a decorative article through or follow with a telescoping technique where the needle may be withdrawn leaving the blanks and transfer pins to insert and guide a decorative article through the pierced tissue.

It should be noted that which the present technology is described in connection with a piercing process, the inventive needle may be used for any other medical uses where it is desired to make an opening through a patient's skin.

The inventive needle may be provided in a variety of sizes—i.e., gauges (G)—depending on the desired application. The gauge refers to the thickness (or outer diameter) of the needle, with smaller numbers indicating thicker needles with a larger outer diameter. When performing a professional body piercing, most piercers will use a hollow needle between 4 and 22 gauge in size. The type of needle used depends on the type of piercing being performed as well as the anatomy of the person being pierced. Examples of various gauges that may be used in accordance with the present invention are summarized below in Table 1.

It is understood that FIGS. 3-4 show only a partial view of the needle at the proximal end. A total length of the needle depends on a particular application. In some embodiments, the needle may be about 1 inch to about 6 inches in total length. The illustration in FIGS. 3-4 should not be interpreted to limit the needle of the present invention to any particular length.

The bottom wall of 106 of the needle 100 terminates at the piercing tip 108. The needle has a primary bevel 120 extending from the bottom of bevel 110 to the piercing tip 108 and a secondary bevel 122 extending from the mid-section 112 to the piercing tip 108. The angle of the primary bevel 120 and the angle of the secondary bevel 122 may be varied depending on a particular application and the overall diameter (gauge) of the needle. In some embodiments, the angle between the primary bevel and the secondary bevel is about 20 degrees to about 30 degrees. The primary bevel 120 has a heel 121 formed by an inner wall edge of the needle where the top wall 104 terminates.

As seen in FIG. 4, the needle body has a bend 119 in the heel 121 area such that the piercing tip 108 is angled toward the top wall 104 of the needle 100. In one exemplary embodiment, the piercing tip 108 is aligned with the top wall 104 such that it extends along a longitudinal axis X that extends along an outer surface of the top wall 104. This alignment enhances the performance and safety of the needles during body piercing procedures, facilitates smoother penetration and minimizes tissue damage during insertion. The bend 119 may be positioned anywhere between the heel 121 and the bottom of the bevel 110 shown in FIG. 3. In some embodiments, the needle body is bent at or adjacent the bottom of the bevel 110. In additional embodiments, the needle body is bent at or adjacent the heel 121. In further embodiments, the needle body is bent at the area between the bottom of the bevel 110 and the heel 121.

In additional exemplary embodiments of the technology, the piercing tip 108 may be bent such that it is positioned below the axis X, but above the heel 121—i.e., above a line formed by an imaginary forward extrapolation of the thickness of the top wall 104 at the heel area 121. When the bend 119 is created, the cutting edge of the heel 121 is pushed downward which also positions it below the piercing tip 108. A bending angle α is an angle between a longitudinal axis Y that extends along an outer surface of the bottom wall 106 distal to the bend 119 and a longitudinal axis Z that extends along an outer surface of the bottom wall 106 of the portion of the needle proximal to the bend 119, as shown in FIG. 4. The bending angle α may be chosen depending on the outer diameter of the needle (gauge), inner diameter, wall thickness, as well as other considerations. In some embodiments, the angle is between about 3°-14°. Exemplary embodiments of different needle gauges and corresponding bend angles are summarized below in Table 1:

| Gauge | OD (inches) | ID (inches) | Degree of Bend |
|---|---|---|---|
| 34RW | .0070-.0075 | .0025-.0040 | 1°-15° |
| 33RW | .0080-.0085 | .0035-.0050 | 1°-15° |
| 32RW | .0090-.0095 | .0035-.0050 | 1°-15° |
| 30TW | .0120-.0125 | .0070-.0080 | 1°-15° |
| 30RW | .0120-.0125 | .0055-.0070 | 1°-15° |
| 29TW | .0130-.0135 | .0080-.0090 | 1°-15° |
| 29RW | .0130-.0135 | .0065-.0080 | 1°-15° |
| 28TW | .0140-.0145 | .0085-.0100 | 1°-15° |
| 28RW | .0140-.0145 | .0065-.0080 | 1°-15° |
| 27XTW | .0160-.0165 | .0115-.0130 | 1°-15° |
| 27TW | .0160-.0165 | .0095-.0110 | 1°-15° |
| 27RW | .0160-.0165 | .0075-.0090 | 1°-15° |
| 26XTW | .0180-.0185 | .0130-.0145 | 1°-15° |
| 26TW | .0180-.0185 | .0115-.0130 | 1°-15° |
| 26RW | .0180-.0185 | .0095-.0110 | 1°-15° |
| 25UTW | .0200-.0205 | .0155-.0170 | 1°-15° |
| 25XTW | .0200-.0205 | .0135-.0150 | 1°-15° |
| 25TW | .0200-.0205 | .0115-.0130 | 1°-15° |
| 25RW | .0200-.0205 | .0095-.0110 | 1°-15° |
| 24UTW | .0220-.0225 | .0175-.0190 | 1°-15° |
| 24XTW | .0220-.0225 | .0155-.0170 | 1°-15° |
| 24TW | .0220-.0225 | .0135-.0155 | 1°-15° |
| 24RW | .0220-.0225 | .0115-.0130 | 1°-15° |
| 23UTW | .0250-.0255 | .0205-.0220 | 1°-15° |
| 23XTW | .0250-.0255 | .0185-.0200 | 1°-15° |
| 23TW | .0250-.0255 | .0165-.0180 | 1°-15° |
| 23RW | .0250-.0255 | .0125-.0140 | 1°-15° |
| 22UTW | .0280-.0285 | .0235-.0250 | 1°-18° |
| 22XTW | .0280-.0285 | .0215-.0230 | 1°-18° |
| 22TW | .0280-.0285 | .0190-.0205 | 1°-18° |
| 22RW | .0280-.0285 | .0155-.0170 | 1°-18° |
| 21UTW | .0320-.0325 | .0265-.0280 | 1°-18° |
| 21XTW | .0320-.0325 | .0245-.0260 | 1°-88° |
| 21TW | .0320-.0325 | .0225-.0240 | 3°-20° |
| 21RW | .0320-.0325 | .0195-.0210 | 3°-20° |
| 20TW | .0355-.0360 | .0255-.0270 | 3°-20° |
| 20RW | .0355-.0360 | .0230-.0245 | 3°-20° |
| 20XTW | .0355-.0360 | .0270-.0285 | 3°-20° |
| 20UTW | .0355-.0360 | .0290-.0305 | 3°-20° |
| 19TW | .0415-.0425 | .0315-.0335 | 3°-20° |
| 19RW | .0415-.0425 | .0255-.0285 | 3°-20° |
| 19XT | .0415-.0425 | .0340-.0360 | 3°-20° |
| 19UTW | .0415-.0425 | .0360-.0380 | 3°-20° |
| 17MT | .0475-.0485 | .0370-.0390 | 3°-20° |
| 18TW | .0495-.0505 | .0375-.0395 | 3°-20° |
| 18RW | .0495-.0505 | .0315-.0345 | 3°-20° |
| 18XTW | .0495-.0505 | .0410-.0430 | 3°-20° |
| 18UTW | .0495-.0505 | .0430-.0450 | 3°-20° |
| 17TW | .0575-.0585 | .0465-.0485 | 3°-20° |
| 17RW | .0575-.0585 | .0405-.0435 | 3°-20° |
| 17XTW | .0575-.0585 | .0490-.0510 | 3°-20° |
| 17UTW | .0575-.0585 | .0510-.0530 | 3°-20° |
| 16TW | .0645-.0655 | .0825-.0545 | 3°-20° |
| 16RW | .0645-.0655 | .0455-.0485 | 3°-20° |
| 16XTW | .0645-.0655 | .0545-.0565 | 3°-20° |
| 16UTW | .0645-.0655 | .0565-.0585 | 3°-20° |
| 15RW | .0715-.0725 | .0525-.0555 | 3°-20° |
| 15TW | .0715-.0725 | .0595-.0615 | 3°-20° |
| 15XTW | .0715-.0725 | .0615-.0635 | 3°-20° |
| 15UTW | .0715-.0725 | .0635-.0655 | 3°-20° |
| 14TW | .0820-.0840 | .0660-.0685 | 3°-20° |
| 14RW | .0820-.0840 | .0610-.0650 | 3°-20° |
| 14XTW | .0820-.0840 | .0710-.0730 | 3°-20° |
| 14UTW | .0820-.0840 | .0730-.0760 | 3°-20° |
| 13TW | .0940-.0960 | .0750-.0790 | 3°-22° |
| 13RW | .0940-.0960 | .0690-.0730 | 3°-22° |
| 13XTW | .0940-.0960 | .0790-.0830 | 3°-22° |
| 12TW | .1080-.1100 | .0890-.0930 | 3°-22° |
| 12RW | .1080-.1100 | .0830-.0870 | 3°-22° |
| 12XTW | .1080-.1100 | .0930-.0970 | 3°-22° |
| 11RW | .1190-.1210 | .0920-.0960 | 3°-22° |
| 11TW | .1190-.1210 | .0980-.1020 | 7°-22° |
| 11XTW | .1190-.1210 | .1020-.1060 | 3°-22° |
| 10TW | .1330-.1350 | .1120-.1160 | 3°-24° |
| 10RW | .1330-.1350 | .1040-.1080 | 3°-24° |
| 10XTW | .1330-.1350 | .1160-.1200 | 3°-24° |
| 9RW | .1470-.1490 | .1150-.1210 | 3°-24° |
| 9TW | .1470-.1490 | .1250-.1310 | 3°-24° |
| 8RW | .1640-.1660 | .1320-.1380 | 3°-24° |
| 8TW | .1640-.1660 | .1420-.1480 | 3°-24° |
| 7RW | .1790-.1810 | .1470-.1530 | 3°-24° |
| 7TW | .1790-.1810 | .1570-.1630 | 3°-24° |
| 6RW | .2020-.2040 | .1700-.1760 | 3°-24° |
| 6TW | .2020-.2040 | .1800-.1860 | 3°-24° |

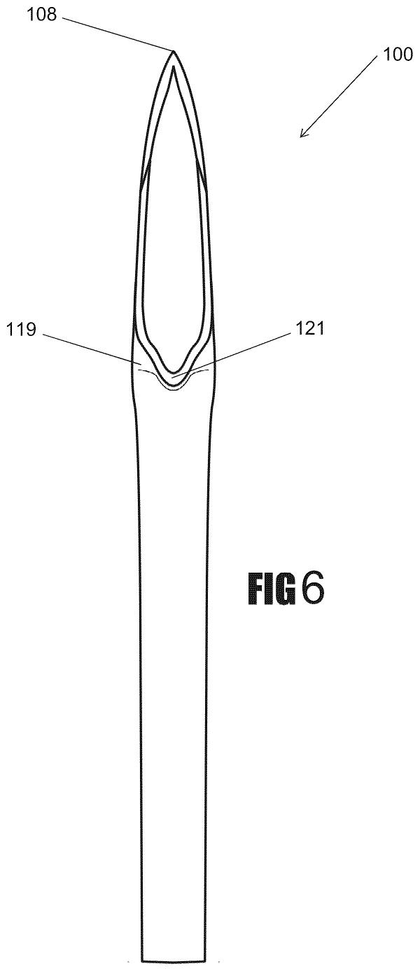

The inventive design of the present technology positions the heel 121 below the piercing tip 108, which prevents the heel 121 from cutting into tissue during piercing, thus minimizing the risk of coring, which optimizes the piercing action by reducing tissue trauma. The bend 119 in the area between the heel 121 and the bottom of the bevel 110 also widens the base of the heel 121 to effectively bypass tissue and further minimize tissue coring. This is best seen in FIGS. 5-6. The inventive design of the needle also enhances user grip and control, eliminating the need for conventional needle-holding techniques.

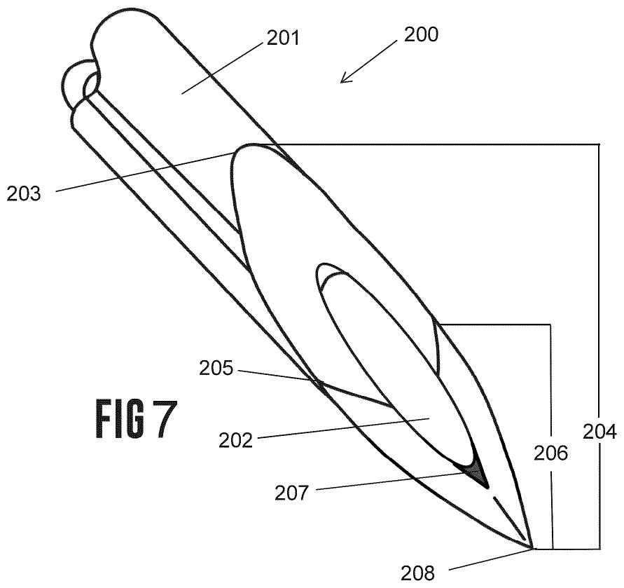

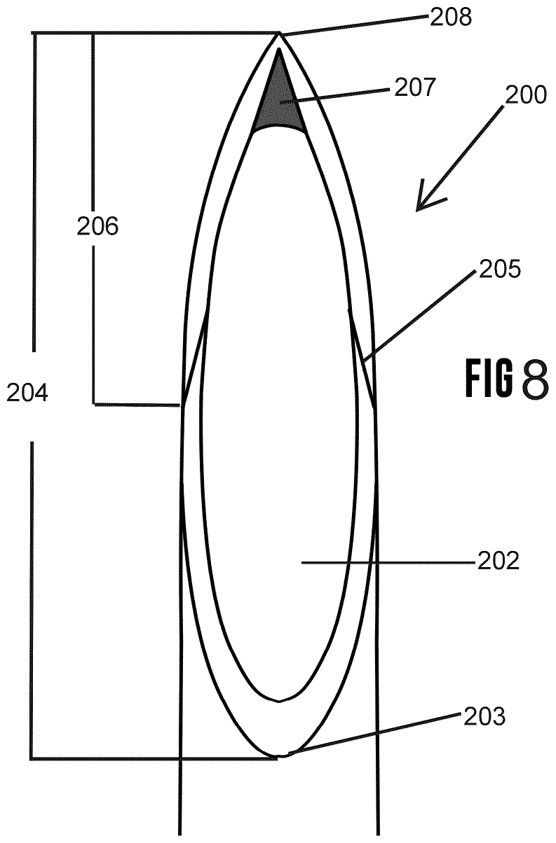

FIGS. 7 and 8 illustrate an exemplary embodiment of a needle tip with a star grind. The needle 200 has a similar construct as described above. The needle 200 has a generally cylindrical body 201 with a hollow piercing channel 202 inside. The needle body terminates at a piercing tip 208. A primary bevel 204 extends from a bottom of bevel 203 to the piercing tip 208 and a secondary bevel 206 extends from a mid-section 205 of the needle to the piercing tip 208. A top surface of the primary bevel 204 and a top surface of the secondary bevel 206 are at an angle to each other, as described above.

The needle further includes a flattened star tip 207, as seen in FIGS. 7-8. The flattened tip 207 has a top surface that extends substantially along the same plane as a plane of a top surface of the first bevel. The top surface is at an angle with respect to the surfaces of the secondary bevel 206 on both sides of the needle. The flattened tip 207 is extends between the piercing tip 208 and an edge of the inner wall of the needle at the tip 208. The flattened tip 207 is produced by a grinding process that minimizes angle transitions between the primary bevel 204 and the secondary bevel 206. By decreasing the secondary bevel angle and reducing the number of passes during the secondary bevel grind, less material is removed in the needle grinding process. This process preserves part of the primary bevel 204 at the flattened tip 207, forming a distinctive star on the top. The inventive star grind creates a flatter cutting surface, optimizing both entry and exit through tissue, which results in a smoother, more controlled transition for improved sharpness. The present inventor has discovered that this design reduces resistance, allowing for a smoother, less traumatic exit through tissue.

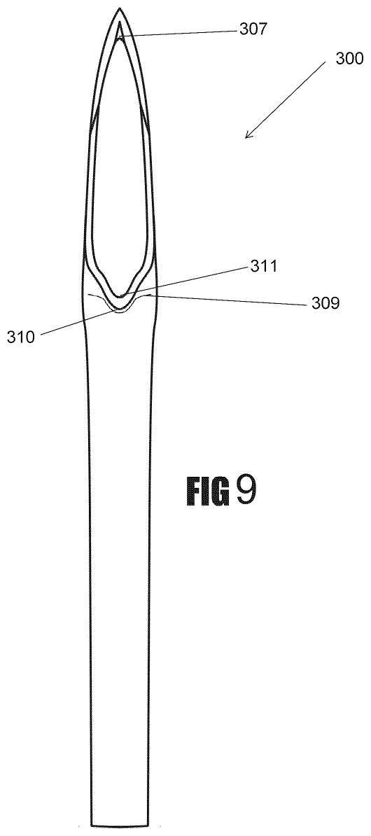

The flattened (star) tip design may be used on needles that do not have the bend, as shown in FIGS. 7-8. It may also be used in conjunction with the bend design as shown in FIG. 9. The needle 300 shown in this figure has a similar design to that shown in FIGS. 3-6 with a bend 309 in the area between the bottom of the bevel 310 and the heel 311, but includes a flattened tip 307, as described above. This inventive design provides for even better piercing process, reducing resistance and preventing tissue damage, while ensuring straight piercing without the need for holding the needle at an angle.

The present technology also comprises method and apparatus for bending needles with a heel bypass bend for medical cosmetology procedures, such as body piercing, or other medical procedures. In one exemplary embodiment, technology uses a press brake unit to precisely bend needles to specific degrees based on the outer diameter (OD) and inner diameter (ID) of the needle tubing. Various suitable press brake units may be used in accordance with the present invention, including but not limited to, a manual press brake, a hydraulic press brake, and a computer numerically controlled (CNC) press brake. The press brake has three basic parts-a ram or top tool, a bottom tool, and a mechanism that drives the ram. In some embodiments, the press brake may have a hydraulic or electrical system that moves the ram and forms the desired shapes and curvatures. In other embodiments, the ram may be hand operated through a crank and shaft.

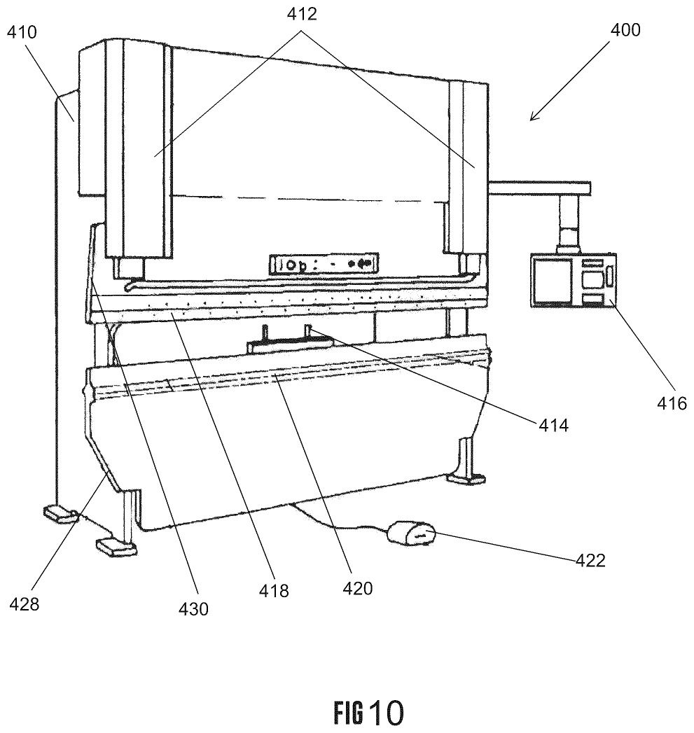

One exemplary embodiment of a CNC press brake is illustrated in FIG. 10. The press brake 400 includes a frame 410 that holds the machine together and supports the other parts of the press brake. A ram mechanism is driven by two synchronized hydraulic cylinders 412 and is a driving mechanism that exerts or resists the force of the press brake. A back gauge 414 is a mechanical system attached to a press brake. The back gauge functions to interact with the press brake computer numerical control, or CNC, and to move along many different axes. The back gauge precisely positions the needle tubing to be bent. The press brake further includes a punch 418 and a die 420. The punch is the top tool, and the die is the bottom tool, and they work together at the processing end of the press brake. The punch and die fold the needle tubing between a matching punch and die and apply force to bend the needle tubing to a predetermined angle. The press brake also includes a toot pedal 422 and a CNC control unit 416 for operation of the system by a user.

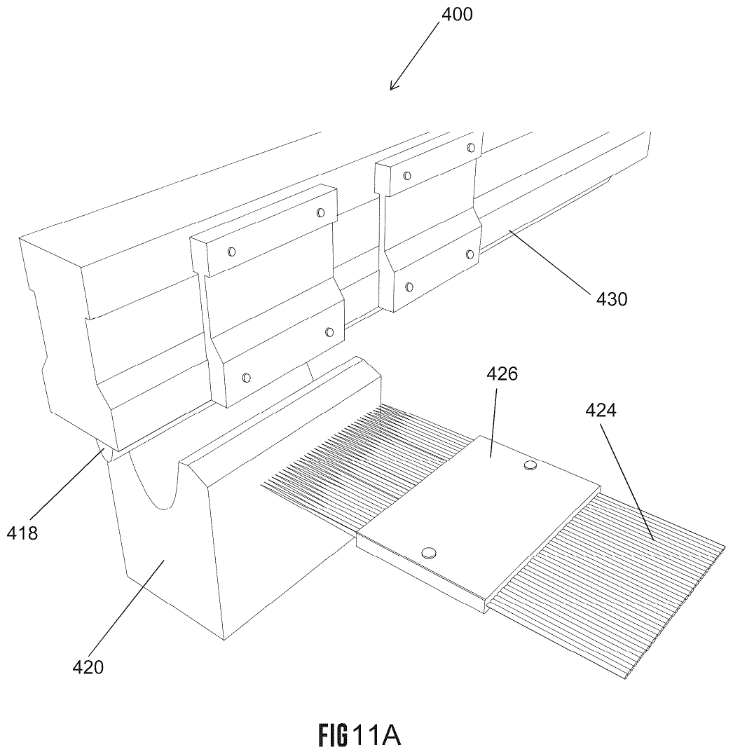

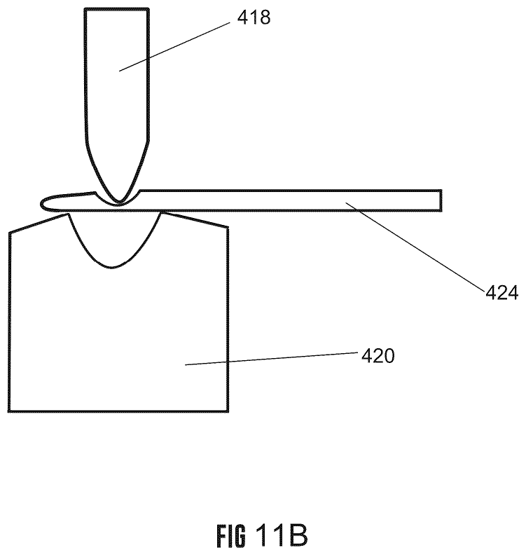

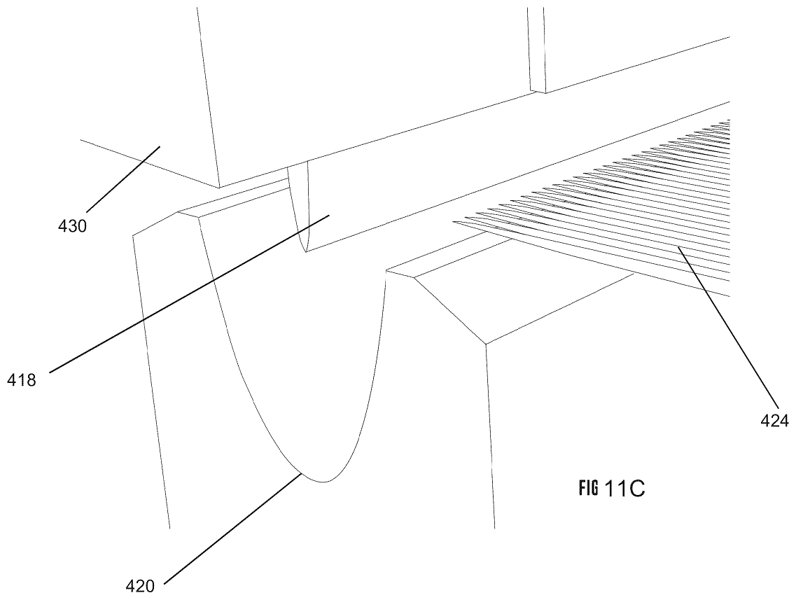

In one exemplary embodiment of the present technology illustrated in FIGS. 11A-11C, the bending process begins with preparing the needles and/or cannula 424, which are held together in strips with filament tape. The strips may be about 5-8 inches in length or may be shorter or longer depending on the particular type of machinery used. The strips may then be securely stabilized in a fixture 426 to prevent movement during bending. Any suitable fixture may be used for this purpose. The press brake unit is then set up with the necessary tooling, tonnage requirements, and programming to accommodate the specific bending requirements of each needle size and degree of bend.

Once set, the fixture 426 with the needles 424 is positioned on the die located on the bed 428 of the press brake unit 400 and aligned with the back gauge 414. The punch 418, attached to the ram 430 of the press brake, is programmed to exert tonnage on the needles 424, thereby bending them into the die 420. As shown in FIG. 11B, the punch 418 is set to bend the needles 424 in heel area set in the CNC control 416 according to a particular size of the needles. The foot pedal 422 controls the bending action, ensuring precision and consistency in the bending process. The punch 418 moves downward toward the fixture 426 with needles 424 using the hydraulic power form the hydraulic cylinders 412. The fixture with needles is positioned such that the edge of the punch 418 comes into contact with the heels of the needles positioned in the fixture to bend the needles in the heel area. When the punch comes into contact with the needles, it will push the needles upward creating a bend. A desired degree of the bend is preprogrammed in the CNC control unit.

The strategic bend created by this process effectively lowers the heel of the needle below the cutting edge, widens the base of the heel to effectively bypass, and aligns the cutting edge with the top of the cannula tubing, as described above. This alignment enhances the performance and safety of the needles during body piercing procedures. Needles with a large outer diameter will require a larger angle of the bend to along the piercing tips of the needles with the top wall of the needles, as discussed above.

The inventive bending process for needles with a heel bypass bend offers significant improvements over traditional methods. By strategically positioning the heel of the needle and aligning the cutting edge with the top of the cannula tubing, this process enhances the functionality and safety of needles used in body piercing applications.

Although the technology has been described and illustrated with respect to exemplary embodiments thereof, it should be understood by those skilled in the art that the foregoing and various other changes, omissions and additions may be made therein and thereto, without parting from the spirit and scope of the present technology. It should also be understood that features described and illustrated in reference to one embodiment may be employed in other embodiments as appropriate.

What is claimed is:

1. A needle, comprising:
an elongated body with a proximal end, a distal end, and a hollow channel extending between the proximal and distal ends, the proximal end configured to penetrate tissue, wherein the elongated body has an upper wall with an inner surface and an outer surface and a bottom wall with an inner surface and an outer surface,
the proximal end comprising a beveled face and an opening within said beveled face in communication with the hollow channel, wherein the beveled face terminates at a piercing tip at its proximal end and has a bevel bottom at its distal end formed by an edge of the outer surface of the top wall,
the beveled face comprising a heel formed by an edge of the inner surface of the top wall delimiting the opening, and
the beveled face comprising a bend positioned between the heel and the bevel bottom such that the piercing tip is positioned above the heel.

2. The needle of claim 1, wherein the piercing tip is aligned with a longitudinal axis of the outer surface of the top wall of the elongated body.

3. The needle of claim 1, wherein the beveled face further comprises a pair of opposing side bevels positioned proximally of the heel, the side bevels intersecting at the piercing tip, wherein a top surface of the side bevels is at an angle to a top surface of the beveled face.

4. The needle of claim 1, wherein the bend has an angle α between a longitudinal axis of the bottom wall of the elongated body distal to the bend and a longitudinal axis of the bottom wall of the elongated body proximal to the bend, wherein a is between about 1 degree to about 24 degrees.

5. The needle of claim 1, further comprising a cannula positioned over the elongated body.

6. The needle of claim 1, wherein the needle is configured for a medical cosmetology procedure.

7. The needle of claim 1, wherein the bend is positioned at the heel.

8. The needle of claim 1, wherein the bend is positioned at the bevel bottom.

9. A needle, comprising:
an elongated body with a proximal end, a distal end, a substantially cylindrical wall, and a hollow channel extending between the proximal and distal ends, the proximal end configured to penetrate tissue;
the proximal end comprising a beveled face and an opening within said beveled face in communication with the hollow channel, wherein the beveled face terminates at a piercing tip at its proximal end;
the beveled face comprising a pair of opposing side bevels intersecting at the piercing tip, wherein a top surface of the side bevels is at an angle to a top surface of the beveled face; and
a star tip positioned adjacent the piercing tip and comprising a top surface extending in the same plane as the top surface of the beveled face and at an angle to a top surface of the side bevels.

10. The needle of claim 9, wherein the top surface of the star tip is substantially flat.

11. The needle of claim 9, wherein the beveled face terminates at a piercing tip at its proximal end and has a bevel bottom at its distal end formed by an edge of an outer surface of the substantially cylindrical wall, the beveled face comprising a heel formed by an edge of an inner surface of the substantially cylindrical wall delimiting the opening, and the beveled face comprising a bend positioned between the heel and the bevel bottom such that the piercing tip is positioned above the heel.

12. The needle of claim 9, wherein the angle between the top surface of the side bevels and the top surface of the beveled face is in a range of about 1 degree to about 24 degrees.

13. A needle, comprising:
an elongated body with a wall and a hollow channel extending therethrough;
a beveled face with an opening within said beveled face in communication with the hollow channel, wherein the beveled face terminates at a piercing tip at its proximal end and has a bevel bottom at its distal end formed by an edge of an outer surface of the wall,
a pair of opposing side bevels intersecting at the piercing tip, wherein a top surface of the side bevels is at an angle to a top surface of the beveled face;
a heel formed by an edge of an inner surface of the wall delimiting the opening, and
a star tip adjacent the piercing tip comprising a top surface extending in the same plane as the top surface of the beveled face and at an angle to a top surface of the side bevels; and
a bend positioned between the heel and the bevel bottom such that the heel is positioned below the piercing tip.

14. The needle of claim 13, wherein the piercing tip is aligned with a longitudinal axis of the outer surface of the wall.

15. The needle of claim 13, wherein the bend has an angle α between a longitudinal axis of the wall distal to the bend and a longitudinal axis of the wall proximal to the bend, wherein a is between about 1 degree to about 24 degrees.

16. The needle of claim 13, wherein the elongated body comprises a widening at the bend.

17. The needle of claim 13, wherein the piercing tip is aligned with a longitudinal axis of the outer surface of the wall opposite the piercing tip.
